(19) United States
(12) Patent Application Publication
(10) Pub. No.: US 2004/0063703 A1
(43) Pub. Date: Apr. 1, 2004

(54) Synergistic pesticide mixtures for the control of animal pests

(76) Inventors: Thomas Bretschneider, Lohmar (DE); Rainer Fuchs, Wuppertal (DE); Wolfram Andersch, Bergisch Gladbach (DE); Ulrich Ebbinghaus-Kintscher, Dortmund (DE); Christoph Erdelen, Leichlingen (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(21) Appl. No.: 10/250,877

(22) PCT Filed: Jan. 7, 2002
(86) PCT No.: PCT/EP02/00059

(30) Foreign Application Priority Data
Jan. 19, 2001 (DE) 101 02 544.0
Jul. 17, 2001 (DE) 101 34 720.0

(51) Int. Cl. A61K 31/538; A61K 31/4439; A61K 31/426

(52) U.S. Cl. 514/229.2; 514/342; 514/341; 514/365

(57) ABSTRACT

The invention relates to synergistic mixtures comprising one or more sodium ion channel effectors and the compound (Z)-3-(6-chloro-3-pyridylmethyl)-1,3-thiazblidin-2-ylidenecyanamide or the compound (E)-1-(2-chloro-1,3-thiazol-5 -ylmethyl)-3-methyl-2-nitroguanidine or the compound 1-[(6-chloro-3-pyridinyl)methyl]-N-nitro-2-imidazolidineimine and the use of these mixtures for controlling animal pests.

The invention relates to synergistic mixtures comprising one or more sodium ion channel effectors and the compound (Z)-3-(6-chloro-3-pyridylmethyl)-1,3-thiazolidin-2-ylidenecyanamide or the compound (E)-1-(2-chloro-1,3-thiazol-5-ylmethyl)-3-methyl-2-nitroguanidine or the compound 1-[(6-chloro-3-pyridinyl)methyl]-N-nitro-2-imidazolidineimine and the use of these mixtures for controlling animal pests.

Mixtures of sodium ion channel antagonists with certain compounds from the group of the modulators of acetylcholine receptors are already known (WO 00/54 591, Research Disclosure May 1997, No. 39786). However, when used, the known mixtures are not always satisfactory, either because the action against certain insects is insufficient or because the required application rates are very high.

It has now been found that mixtures comprising one or more sodium ion channel effectors and the compound (Z)-3-(6-chloro-3-pyridylmethyl)-1,3-thiazolidin-2-ylidenecyanamide (thiacloprid) or the compound (E)-1-(2-chloro-1,3-thiazol-5-yl-methyl)-3-methyl-2-nitroguanidine (chlothianidin) or the compound 1-[(6-chloro-3-pyridinyl)methyl]-N-nitro-2-imidazolidineimine (imidacloprid) have synergistic action and are suitable for controlling animal pests. Owing to this synergism, it is possible to use considerably lower amounts of active compounds, i.e. the activity of the mixture is higher than the activity of the individual components.

The term sodium ion channel effector refers to a compound which prevents the transport of sodium ions across the cell membrane of a nerve cell. Such compounds and their mode of action are described, for example, in “Pesticide Biochemistry and Physiology, 60: 177-185” and “Archives of Insect Biochemistry and Physiology, 37: 91-103”. Sodium ion channel inhibitors are described, for example, in the U.S. Pat. No. 5,543,573; U.S. Pat. No. 5,708,170; U.S. Pat. No. 5,324,837 and U.S. Pat. No. 5,462,938. By way of example, compounds of the following formulae may be mentioned:

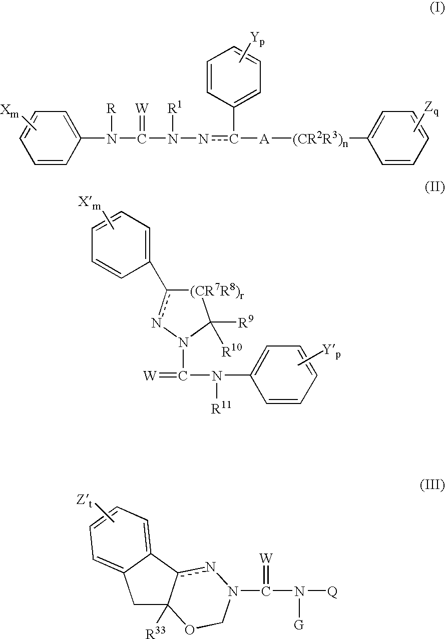

in which

A represents CR⁴R⁵ or NR⁶,

W represents O or S,

X, Y, Z, X′, Y′ and Z′ independently of one another represent H, halogen, OH, CN, NO₂,

represent C₁-C₆-alkyl which is optionally mono- or polysubstituted by halogen, C₁-C₃-alkoxy, C₁-C₃-halogenoalkoxy, C₃-C₆-cycloalkyl, C₂-C₆-alkenyloxy or sulphonyloxy,

represents C₁-C₆-alkoxy which is optionally mono- or polysubstituted by halogen, C₁-C₃-alkoxy or C₃-C₆-cycloalkyl,

represents C₁-C₆-alkoxycarbonyl, represents C₃-C₆-cycloalkylcarbonyloxy, represents phenyl which is optionally mono- or polysubstituted by halogen, C₁-C₄-alkyl or C₁-C₄-alkoxy,

represents aminocarbonyloxy which is optionally mono- or disubstituted by C₁-C₃-alkyl,

represents C₁-C₆-alkoxycarbonyloxy, represents C₁-C₆-alkylsulphonyloxy, represents C₂-C₆-alkenyl or represents NR₁₂R₁₃,

m, p and q independently of one another represent 1, 2, 3, 4 or 5,

n represents 0, 1 or 2,

r represents 1 or 2,

t represents 1, 2, 3 or 4,

R, R¹, R², R³, R⁴ and R⁵ independently of one another represent H or C₁-C₄-alkyl,

R⁶ represents H, C₁-C₆-alkyl, C₁-C₆-halogenoalkyl, C₁-C₆-alkoxyalkyl, C₁-C₆-alkoxy, C₁-C₆-halogenoalkoxy, C₂-C₆-alkenyl, C₂-C₆-alkinyl, C₁-C₆-alkyl-carbonyl, C₁-C₆-alkoxycarbonyl, C₁-C₆-alkylthio or C₁-C₆-halogeno-alkylthio,

R⁷ and R⁸ independently of one another represent H, halogen, C₁-C₆-alkyl, C₁-C₆-alkylcarbonyloxy or represent phenyl which is optionally mono- or polysubstituted by halogen, CN, NO₂, C₁-C₆-alkyl, C₁-C₆-halogenoalkyl, C₁-C₆-alkoxy or C₁-C₆-halogenoalkoxy,

R⁹ and R¹⁰ independently of one another represent H or C₁-C₄-alkyl,

R¹¹ represents H, C₁-C₆-alkyl, C₁-C₆-halogenoalkyl, C₁-C₄-alkylcarbonyl, C₁-C₆-alkoxycarbonyl or C₁-C₆-halogenoalkoxycarbonyl,

R¹² and R¹³ independently of one another represent H or C₁-C₆-alkyl,

G represents H,

represents C₁-C₆-alkyl which is optionally mono- or polysubstituted by halogen, C₁-C₄-alkoxy, C₁-C₆-halogenoalkoxy, CN, NO₂, S(O)ₙR¹⁴, COR¹⁵, CO₂R¹⁶, phenyl or C₃-C₆-cycloalkyl,

represents C₁-C₆-alkoxy, C₁-C₆-halogenoalkoxy, CN, NO₂, S(O)ₙR¹⁷, COR¹⁸, CO₂R¹⁹,

represents phenyl which is optionally mono- or polysubstituted by halogen, CN, C₁-C₃-halogenoalkyl or C₁-C₃-halogenoalkoxy,

represents C₃-C₆-cycloalkyl or phenylthio,

Q represents phenyl which is optionally mono- or polysubstituted by halogen, CN, SCN, NO₂, S(O)ₙR²⁰, C₁-C₄-alkyl, C₁-C₄-halogenoalkyl, C₁-C₄-alkoxyalkyl, C₁-C₆-alkoxy, C₁-C₆-halogenoalkoxy or NR²¹R²²,

n represents 0, 1 or 2,

R¹⁴, R¹⁵, R¹⁶, R¹⁸, R¹⁹, R²¹ and R²² independently of one another represent H or C₁-C₆-alkyl,

R¹⁷ and R²⁰ independently of one another represent C₁-C₆-alkyl or C₁-C₆-halogenoalkyl,

R³³ represents CO₂R³⁴,

R³⁴ represents H, C₁-C₆-alkyl, C₁-C₆-halogenoalkyl, phenyl or halogenophenyl and the group

C [symbol] N represents C—N or, in particular, C═N.

Preference is given to sodium ion channel effectors of the formula (I), in particular to those in which C [symbol] N represents C═N.

Preference is furthermore given to sodium ion channel effectors of the formula (II), in particular to those in which C [symbol] N represents C═N.

Preference is furthermore given to sodium ion channel effectors of the formula (III), in particular to those in which C [symbol] N represents C═N.

Preference is given to compounds of the formula (I) in which

Xₘ represents 4-OCF₃ or 4-SCF₃,

Yₚ represents 3-Cl, 3-CF₃, 3-CN, 4-Cl, 4-CF₃ or 4-CN and

Z_{q} represents 3-Cl, 3-CF₃, 3-CN, 4-Cl, 4-CF₃ or 4-CN.

Mention may be made of the compound of the formula

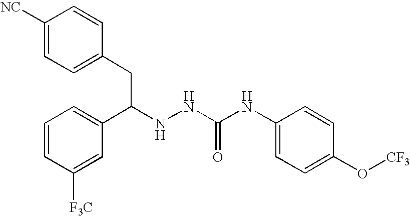

Preference is given to those compounds of the formula (II) in which

Y′ₚ represents 4-OCF₃ or 4-SCF₃,

X′ₘ represents 3-Cl, 3-CF₃, 3-CN, 4-Cl, 4-CF₃ or 4-CN,

R⁷ represents H and

R⁸ represents phenyl which is substituted by 3-Cl, 3-CF₃, 3-CN, 4-Cl, 4-CF₃ or 4-CN.

Mention may be made of the compound of the formula

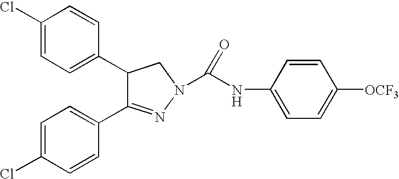

Preference is given to those compounds of the formula (III) in which

Cl represents phenyl which is substituted by 4-OCF₃ or 4-SCF₃,

G represents H, CO₂CH₃ or CO₂C₂H₅,

R³³ represents CO₂CH₃ or CO₂C₂H₅ and

Z′ₜ represents 3-Cl, 3-CF₃, 3-CN, 4-Cl, 4-CF₃ or 4-CN.

Mention may be made of the compound of the formula

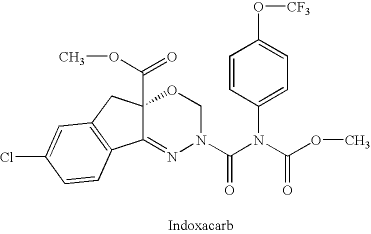

Further sodium ion channel inhibitors are mentioned in U.S. Pat. No. 5,116,850 and U.S. Pat. No. 5,304,573. By way of example, compounds of the following formulae may be listed

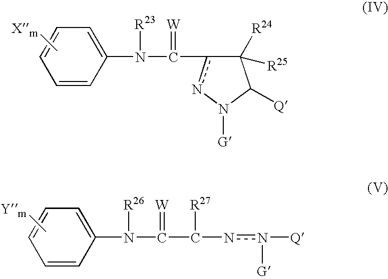

in which

W represents oxygen or sulphur,

X″ and Y″ independently of one another represent H, CN, SCN,

represent C₁-C₆-alkyl which is optionally mono- or polysubstituted by halogen, NO₂, CN, C₁-C₄-alkoxy, C₁-C₄-alkylthio, phenyl, halogenophenyl, C₁-C₄-alkylsulphonyl or C₁-C₄-alkoxycarbonyl,

represent C₂-C₄-alkenyl, C₂-C₄-halogenoalkenyl, C₂-C₄-alkinyl, C₂-C₄-halogenoalkinyl, C₃-C₆-cycloalkyl, C₃-C₆-halogenocycloalkyl,

represent phenyl which is optionally mono- or polysubstituted by halogen, CN, NO₂, C₁-C₄-alkyl, C₁-C₄-halogenoalkyl, C₁-C₄-alkoxy, C₁-C₄-halogenoalkoxy, C₁-C₄-alkylthio, C₁-C₄-alkylsulphonyl or C₁-C₄-halogenoalkylsulphonyl,

represent a six-membered aromatic ring which contains one or two heteroatoms, where these heteroatoms are selected from the group consisting of 0 or 1 oxygen atom, 0 or 1 sulphur atom and 0, 1 or 2 nitrogen atoms and where this heteroaromatic ring is attached via a carbon atom and is optionally mono- or polysubstituted by the groups mentioned under “X”,

Q′ represents H,

represents C₁-C₆-alkyl which is optionally mono- or polysubstituted by halogen, CN, C₁-C₃-alkoxy, C₁-C₆-alkoxycarbonyl or phenyl, where phenyl for its part may be mono- or polysubstituted by halogen, CN, NO₂, C₁-C₄-alkyl, C₁-C₄-halogenoalkyl, C₁-C₄-alkylsulphonyl and C₁-C₄-alkylsulphinyl,

represents C₂-C₆-alkenyl, C₂-C₆-alkinyl or

represents C₁-C₄-alkylcarbonyl, C₁-C₄-halogenoalkylcarbonyl or represents NR²⁸R²⁹, or

represents a five- or six-membered heteroaromatic ring (for example pyridyl) which contains one or two heteroatoms, where these heteroatoms are selected from the group consisting of 0 or 1 oxygen atom, 0 or 1 sulphur atom and 0, 1 or 2 nitrogen atoms and where this heteroaromatic ring is attached via a carbon atom and is optionally mono- or polysubstituted by the groups mentioned under X″,

m represents 0, 1, 2, 3, 4 or 5,

G′ represents phenyl which is optionally mono- or polysubstituted by the groups mentioned under X″,

represents a five- or six-membered heteroaromatic ring (for example pyridyl) which contains one or two heteroatoms, where these heteroatoms are selected from the group consisting of 0 or 1 oxygen atom, 0 or 1 sulphur atom and 0, 1 or 2 nitrogen atoms and where this heterocyclic ring is attached via a carbon atom and is optionally mono- or polysubstituted by the groups mentioned under X″,

R²³, R²⁴, R²⁵, R²⁶, R²⁷, R²⁸ and R²⁹ independently of one another represent H or C₁-C₄-alkyl and the group

C [symbol] N represents C—N or, in particular, C═N.

Preference is given to those compounds of the formula (IV), in which

X″ₘ represents 4-OCF₃ or 4-SCF₃,

G′ represents phenyl which is substituted by 3-Cl, 3-CF₃, 3-CN, 4-Cl, 4-CF₃ or 4-CN and

Q′ represents a heterocycle which is substituted by C₁ or CF₃, for example 4-C₁₋₂-pyridyl.

By way of example, the following compound may be mentioned:

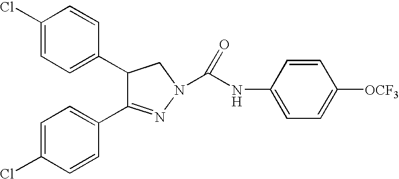

Preference is given to those compounds of the formula (V), in which

Y″ₘ represents 4-OCF₃ or 4-SCF₃.

By way of example, the following compound may be mentioned:

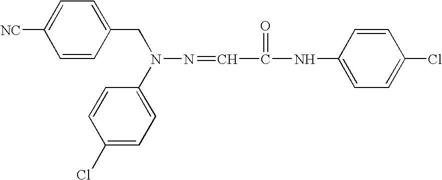

Further sodium ion channel effectors are benzophenone-hydrazones which are known, for example, from EP-O-0 742 202, JP-1000 14 69, WO 96/33 1.68, WP-O-647 622, WO 97/11 050 and WO 97/38 973. These include the benzophenone-hydrazones of the formula (VI)

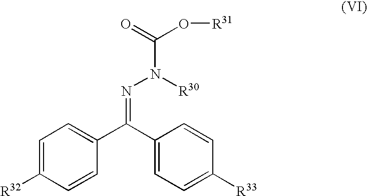

in which

R³⁰ represents H, C₁-C₆-alkyl or C₁-C₆-alkoxycarbonyl, in particular H or COCH₃,

R³¹ represents C₁-C₆-alkyl, in particular methyl or ethyl,

R³² represents halogen, in particular chlorine, and

R³³ represents O—SO₂—C₁-C₆-alkyl or O—SO₂—C₁-C₆-halogenoalkyl, in particular O—SO₂CH₃ or O—SO₂—CF₃, or

represents CH₂—S(O)_{y}—C₁-C₆-alkyl or CH₂—S(O)_{y}—C₁-C₆-halogenoalkyl, in which

y represents 0, 1 or 2,

represents in particular (CH₂)S(O)_{y}CH₃, CH₂—S(O)_{y}C₂H₅ or CH₂—S(O)_{y}CH₂CF₃, especially preferably CH₂—S—CH₃.

The following compounds may be specifically mentioned:

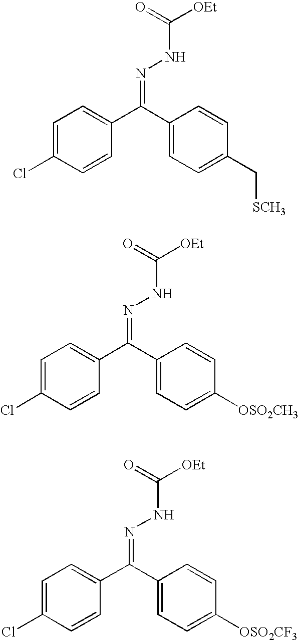

A further group of sodium ion channel inhibitors are bis-aryl-methylpiperidines which are known, for example, from WO 95/23 507, U.S. Pat. No. 5,569,664, WO 96/36 228, WO 97/26 252, WO 98/00 015, WO 99/14 193 and WO 00/01 838. These include the compounds of the formula (VII)

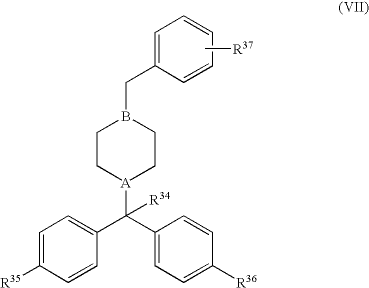

in which

R³⁴ represents H or OH,

R³⁵ and R³⁶ independently of one another represent halogen, C₁-C₆-halogenoalkyl, halogenoalkylthio or C₁-C₆-halogenoalkoxy, in particular CF₃, OCF₃ and SCF₃,

R³⁷ represents H, C₁-C₆-alkyl, C₁-C₆-halogenoalkyl, C₁-C₆-alkoxy, C₁-C₆-halogenoalkoxy, C₁-C₆-alkoxycarbonyl, NH—CO₂—C₁-C₆-alkyl or CH═N—O—C₁-C₆-alkyl,

A represents CH or N and

B represents N or N^{⊕}—O^{⊖}.

The following compounds may be specifically mentioned:

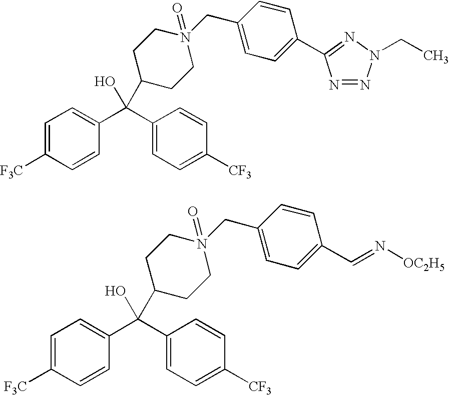

A further group of sodium ion channel effectors are oxadiazines which are known, for example, from WO 96/36 618, WO 99/41 245, U.S. Pat. No. 5,536,720 and WO 98/33 794.

These include the compounds of the formula (VIII)

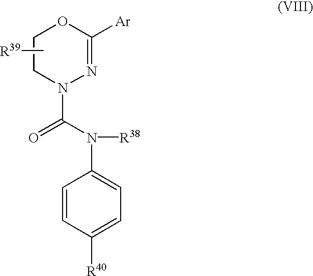

in which

R³⁸ represents H, C₁-C₆-alkyl, C₁-C₆-alkylcarbonyl or C₁-C₆-alkylcarbonyloxy, in particular H,

R³⁹ represents C₁-C₆-alkyl or C₁-C₆-alkoxy,

R⁴⁰ represents halogen, C₁-C₆-halogenoalkyl, C₁-C₆-halogenoalkoxy or C₁-C₆-halogenoalkylthio, in particular chlorine, CF₃, OCF₃ or SCF₃, and

Ar represents phenyl which is optionally mono- or polysubstituted by halogen, CN, C₁-C₆-alkyl or C₁-C₆-halogenoalkyl, in particular 4-chlorophenyl, 4-cyanophenyl, 4-trifluoromethylphenyl, 3-chlorophenyl, 3-cyanophenyl or 3-trifluoromethylphenyl, or

represents 5- or 6-membered hetaryl having in each case one O, S or N atom, in particular thienyl, furanyl or pyridyl, optionally mono- or polysubstituted by halogen, CN, C₁-C₆-alkyl or C₁-C₆-halogenoalkyl.

The following compounds may be mentioned by way of example:

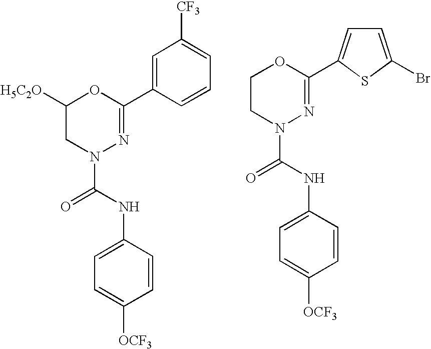

In addition to one or more compounds from the group of the sodium ion channel effectors disclosed above, the mixtures according to the invention comprise at least one of the following compounds of the formulae (IX), (X) and (XI)

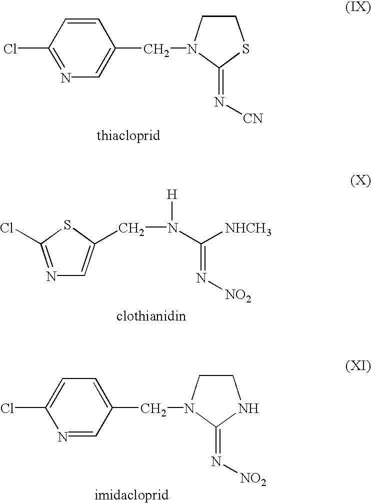

Compound (IX) is known from U.S. Pat. No. 4,849,432.

Compound (X) is known from EP-O-375 907-A1.

Compound (XI) is known from EP-O-192 060.

The ratio (in parts by weight) of Na ion channel effectors to compounds of the formulae (IX) to (XI) in the mixtures is generally from 1:0.1 to 1:100, preferably from 1:1 to 1:50.

A particularly preferred mixture according to the invention comprises the active compounds indoxacarb of the formula

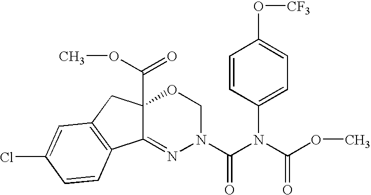

and clothianidin of the formula (X)

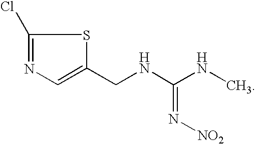

A further particularly preferred mixture according to the invention comprises the active compounds indoxacarb of the formula

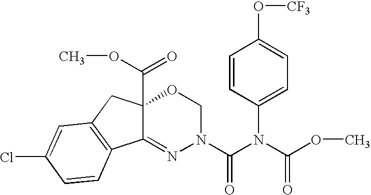

and thiacloprid of the formula (IX)

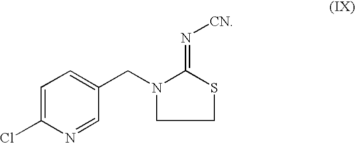

A further particularly preferred mixture according to the invention comprises the active compounds indoxacarb of the formula

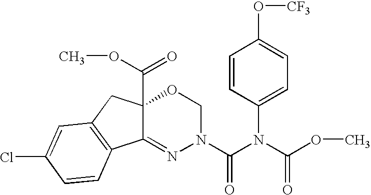

and imidacloprid of the formula (XI)

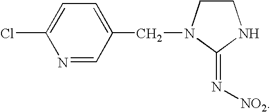

The active compound combinations (mixtures) are suitable for controlling animal pests, in particular insects, arachnids and nematodes, found in agriculture, in forests, in the protection of stored products and materials and in the hygiene sector, and they are tolerated well by plants and have favourable homeotherm toxicity. They can preferably be used as crop protection compositions. They are active against normally sensitive and resistant species, and against all or individual developmental stages. The abovementioned pests include:

From the order of the Isopoda, for example, Oniscus asellus, Armadillidium vulgare, Porcellio scaber.

From the order of the Diplopoda, for example, Blaniulus guttulatus.

From the order of the Chilopoda, for example, Geophilus carpophagus, Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp., Schistocerca gregaria.

From the order of the Blattaria, for example, Blatta orientalis, Periplaneta americana, Leucophaea maderae, Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp., Damalinia spp.

From the order of the Thysanoptera, for example, Hercinothrips femoralis, Thrips tabaci, Thrips palmi, Frankliniella accidentalis.

From the order of the Heteroptera, for example, Eurygaster spp., Dysdercus intermedius, Piesma quadrata, Cimex lectularius, Rhodnius prolixus, Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp.

From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica, Lissorhoptrus oryzophilus.

From the order of the Hymenoptera, for example, Diprion spp., Hoplocampa spp., Lasius spp., Monomorium pharaonis, Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp., Liriomyza spp.

From the order of the Siphonaptera, for example, Xenopsylla cheopis, Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

According to the invention, it is possible to treat all plants and parts of plants. Plants are to be understood here as meaning all plants and plant populations such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including the plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested plants and vegetative and generative propagation material, for example seedlings, tubers, rhizomes, cuttings and seeds.

The treatment according to the invention of the plants and parts of plants with the active compounds is carried out directly or by action on their environment, habitat or storage area according to customary treatment methods, for example by dipping, spraying, evaporating, atomizing, broadcasting, brushing-on and, in the case of propagation material, in particular in the case of seeds, furthermore by one- or multi-layer coating.

The active compound combinations can be converted into the customary formulations such as solutions, emulsions, wettable powders, suspensions, powders, dusts, pastes, soluble powders, granules, suspension-emulsion concentrates, natural and synthetic materials impregnated with active compound, and microencapsulations in polymeric materials.

These formulations are produced in a known manner, for example by mixing the active compounds with extenders, that is, liquid solvents and/or solid carriers, optionally with the use of surfactants, that is, emulsifiers and/or dispersants, and/or foam formers.

If the extender used is water, it is also possible, for example, to use organic solvents as cosolvents. The following are essentially suitable as liquid solvents: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water.

Suitable solid carriers are:

for example ammonium salts and ground natural minerals such as kaolins, clays, talc, chalk, quartz, attapulgite, montmorillonite or diatomaceous earth, and ground synthetic materials such as highly-disperse silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates; suitable dispersants are: for example lignin-sulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other additives can be mineral and vegetable oils.

It is possible to use colorants such as inorganic pigments, for example iron oxide, titanium oxide and Prussian Blue, and organic colorants such alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

The active compound according to the invention can be present in its commercially available formulations and in the use forms, prepared from these formulations, as a mixture with other active compounds, such as insecticides, attractants, sterilants, bactericides, acaricides, nematicides, fungicides, growth-regulating substances or herbicides. The insecticides include, for example, phosphates, carbamates, carboxylates, chlorinated hydrocarbons, phenylureas and substances produced by microorganisms, inter alia.

Particularly advantageous co-components are, for example, the following:

Fungicides:

aldimorph, ampropylfos, ampropylfos-potassium, andoprim, anilazine, azaconazole, azoxystrobin,

benalaxyl, benodanil, benomyl, benzamacril, benzamacril-isobutyl, bialaphos, binapacryl, biphenyl, bitertanol, blasticidin-S, bromuconazole, bupirimate, buthiobate,

calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram,

debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,

edifenphos, epoxiconazole, etaconazole, ethirimol, etridiazole,

famoxadon, fenapanil, fenarimol, fenbuconazole, fenfuram, fenitropan, fenpiclonil, fenpropidin, fenpropimorph, fentin acetate, fentin hydroxide, ferbam, feiimzone, fluazinam, flumetover, fluoromide, fluquinconazole, flurprimidol, flusilazole, flusulphamide, flutolanil, flutriafol, folpet, fosetyl-aluminium, fosetyl-sodium, fthalide, fuberidazole, furalaxyl, furametpyr, furcarbonil, furconazole, furconazole-cis, funnecyclox,

guazatine, hexachlorobenzene, hexaconazole, hymexazole,

imazalil, imibenconazole, iminoctadine, iminoctadine albesilate, iminoctadine triacetate, iodocarb, ipconazole, iprobenfos (IBP), iprodione, irumamycin, isoprothiolane, isovaledione,

kasugamycin, kresoxim-methyl, copper preparations, such as: copper hydroxide, copper naphthenate, copper oxychloride, copper sulphate, copper oxide, oxine-copper and Bordeaux mixture,

mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,

nickel dimethyldithiocarbamate, nitrothal-isopropyl, nuarimol,

ofurace, oxadixyl, oxamocarb, oxolinic acid, oxycarboxim, oxyfenthiin,

paclobutrazole, pefurazoate, penconazole, pencycuron, phosdiphen, pimaricin, piperalin, polyoxin, polyoxorim, probenazole, prochloraz, procymidone, propamocarb, propanosine-sodium, propiconazole, propineb, pyrazophos, pyrifenox, pyrimethanil, pyroquilon, pyroxyfur,

quinconazole, quintozene (PCNB),

sulphur and sulphur preparations,

tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, triflumizole, triforine, triticonazole,

uniconazole,

validamycin A, vinclozolin, viniconazole,

zarilamide, zineb, ziram and also

Dagger G,

OK-8705, OK-8801,

α-(1,1-dimethylethyl)-β-(2-phenoxyethyl)-1H-1,2,4-triazole-1-ethanol,

α-(2,4-dichlorophenyl)-β-fluoro-β-propyl-1H-1,2,4-triazole-1-ethanol,

α-(2,4-dichlorophenyl)-β-methoxy-α-methyl-1H-1,2,4-triazole-1-ethanol,

α-(5-methyl-1,3-dioxan-5-yl)-β-[[4-(trifluoromethyl)-phenyl]-methylene]-1H-1,2,4-triazole-1-ethanol,

(5RS,6RS)-6-hydroxy-2,2,7,7-tetramethyl-5-(1H-1,2,4-triazol-1-yl)-3-octanone,

(E)-α-(methoxyimino)-N-methyl-2-phenoxy-phenylacetamide,

1-isopropyl {2-methyl-1-[[[1-(4-methylphenyl)-ethyl]-amino]-carbonyl]-propyl}-carbamate,

1-(2,4-dichlorophenyl)-2-(1H-1,2,4-triazol-1-yl)-ethanone-O-(phenylmethyl)-oxime,

1-(2-methyl-1-naphthalenyl)-1H-pyrrole-2,5-dione,

1-(3,5-dichlorophenyl)-3-(2-propenyl)-2,5-pyrrolidinedione,

1-[(diiodomethyl)-sulphonyl]-4-methyl-benzene,

1-[[2-(2,4-dichlorophenyl)-1,3-dioxolan-2-yl)-methyl]-1H-imidazole,

1-[[2-(4-chlorophenyl)-3-phenyloxiranyl]-methyl]-1H-1,2,4-triazole,

1-[1-[2-[(2,4-dichlorophenyl)-methoxy]-phenyl]-ethenyl]-1H-imidazole,

1-methyl-5-nonyl-2-(phenylmethyl)-3-pyrrolidinole,

2′,6′-dibromo-2-methyl4′-trifluoromethoxy4′-trifluoro-methyl-1,3-thiazole-5-carboxanilide,

2,2-dichloro-N-[1-(4-chlorophenyl)-ethyl]-1-ethyl-3-methyl-cyclopropanecarboxamide,

2,6-dichloro-5-(methylthio)₄-pyrimidinyl-thiocyanate,

2,6-dichloro-N-(4-trifluoromethylbenzyl)-benzamide,

2,6-dichloro-N-[4-(trifluoromethyl)-phenyl]-methyl]-benzamide,

2-(2,3,3-triiodo-2-propenyl)-2H-tetrazole,

2-[(1-methylethyl)-sulphonyl]-5-(trichloromethyl)-1,3,4-thiadiazole,

2-[[6-deoxy4-O-(4-O-methyl-β-D-glycopyranosyl)-α-D-glucopyranosyl]-amino]4-methoxy-1H-pyrrolo[2,3-d]pyrimidine-5-carbonitrile,

2-aminobutane,

2-bromo-2-(bromomethyl)-pentanedinitrile,

2-chloro-N-(2,3-dihydro-1,1,3-trimethyl-1H-inden-4-yl)-3-pyridinecarboxamide,

2-chloro-N-(2,6-imethylphenyl)-N-(isothiocyanatomethyl)-acetamide,

2-phenylphenol (OPP),

3,4-dichloro-1-[4-(difluoromethoxy)-phenyl]-1H-pyrrole-2,5-dione,

3,5-dichloro-N-[cyano[(1-methyl-2-propynyl)-oxy]-methyl]-benzamide,

3-(1,1-dimethylpropyl-1-oxo-1H-indene-2-carbonitrile,

3-[2-(4-chlorophenyl)-5-ethoxy-3-isoxazolidinyl]-pyridine,

4-chloro-2-cyano-N,N-dimethyl-5-(4-methylphenyl)-1H-imidazole-1-sulphonamide,

4-methyl-tetrazolo[1,5-a]quinazolin-5(4H)-one,

8-(1,1-dimethylethyl)-N-ethyl-N-propyl-1,4-dioxaspiro[4.5]decane-2-methanamine,

8-hydroxyquinoline sulphate,

9H-xanthene-2-[(phenylamino)-carbonyl]-9-carboxylic hydrazide,

bis-(1-methylethyl)-3-methyl-4-[(3-methylbenzoyl)-oxy]-2,5-thiophenedicarboxylate,

cis-1-(4-chlorophenyl)-2-(1H-1,2,4triazol-1-yl)-cycloheptanol,

cis-4-[3-[4-(1,1-dimethylpropyl)-phenyl-2-methylpropyl]-2,6-dimethyl-morpholine hydrochloride,

ethyl [(4-chlorophenyl)-azo]-cyanoacetate,

potassium bicarbonate,

methanetetrathio]-sodium salt,

methyl 1-(2,3-dihydro-2,2-dimethyl-1H-inden-1-yl)-1H-imidazole-5-carboxylate,

methyl N-(2,6-dimethylphenyl)-N-(5-isoxazolylcarbonyl)-DL-alaninate,

methyl N-(chloroacetyl)-N-(2,6-dimethylphenyl)-DL-alaninate,

N-(2,3-dichloro-4-hydroxyphenyl)-1-methyl-cyclohexanecarboxamide,

N-(2,6-dimethylphenyl)-2-methoxy-N-(tetrahydro-2-oxo-3-furanyl)-acetamide,

N-(2,6-dimethylphenyl)-2-methoxy-N-(tetrahydro-2-oxo-3-thienyl)-acetamide,

N-(2-chloro-4-nitrophenyl)₄-methyl-3-nitro-benzenesulphonamide,

N-(4-cyclohexylphenyl)-1,4,5,6-tetrahydro-2-pyrimidinamine,

N-(4-hexylphenyl)-1,4,5,6-tetrahydro-2-pyrimidinamine,

N-(5-chloro-2-methylphenyl)-2-methoxy-N-(2-oxo-3-oxazolidinyl)-acetamide,

N-(6-methoxy-3-pyridinyl)-cyclopropanecarboxamide,

N-[2,2,2-trichloro-1-[(chloroacetyl)-amino]-ethyl]-benzamide,

N-[3-chloro-4,5-bis(2-propinyloxy)-phenyl]-N′-methoxy-methanimidamide,

N-formyl-N-hydroxy-DL-alanine-sodium salt,

O,O-diethyl [2-(dipropylamino)-2-oxoethyl]-ethylphosphoramidothioate,

O-methyl S-phenyl phenylpropylphosphoramidothioate,

S-methyl 1,2,3-benzothiadiazole-7-carbothioate,

spiro[2H]-1-benzopyran-2,1′(3′H)-isobenzofuran-3′-one

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:

abamectin, acephate, acetamiprid, acrinathrin, alanycarb, aldicarb, aldoxycarb, alpha-cypermethrin, alphamethrin, amitraz, avermectin, AZ 60541, azadirachtin, azamethiphos, azinphos A, azinphos M, azocyclotin,

Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, BPMC, bromophos A, bufencarb, buprofezin, butathiofos, butocarboxim, butylpyridaben,

cadusafos, carbaryl, carbofuran, carbophenothion, carbosulfan, cartap, chloethocarb, chlorethoxyfos, chlorfenapyr, chlorfenvinphos, chlorfluazuron, chlornephos, chlorpyrifos, chlorpyrifos M, chlovaporthrin, cis-resmethrin, cispermethrin, clocythrin, cloethocarb, clofentezine, cyanophos, cycloprene, cycloprothrin, cyfluthrin, cyhalothrin, cyhexatin, cypermethrin, cyromazine,

deltamethrin, demeton M, demeton S, demeton-S-methyl, diafenthiuron, diazinon, dichlorvos, diflubenzuron, dimethoate, dimethylvinphos, diofenolan, disulfoton, docusat-sodium, dofenapyn,

eflusilanate, emamectin, empenthrin, endosulfan, Entomopfthora spp., eprinomectin, esfenvalerate, ethiofencarb, ethion, ethoprophos, etofenprox, etoxazole, etrimfos,

fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazinam, fluazuron, flubrocythrinate, flucycloxuron, flucythrinate, flufenoxuron, flutenzine, fluvalinate, fonophos, fosmethilan, fosthiazate, fubfenprox, furathiocarb,

granulosis viruses,

halofenozide, HCH, heptenophos, hexaflumuron, hexythiazox, hydroprene,

imidacloprid, isazofos, isofenphos, isoxathion, ivernectin,

nuclear polyhedrosis viruses,

lambda-cyhalothrin, lufenuron,

malathion, mecarbam, metaldehyde, methamidophos, Metharhizium anisopliae, Metharhizium flavoviride, methidathion, methiocarb, methomyl, methoxyfenozide, metolcarb, metoxadiazone, mevinphos, milbemectin, monocrotophos,

naled, nitenpyram, nithiazine, novaluron,

omethoate, oxamyl, oxydemethon M,

Paecilomyces fumosoroseus, parathion A, parathion M, permethrin, phenthoate, phorate, phosalone, phosmet, phosphamidon, phoxim, pirimicarb, pirimiphos A, pirimiphos M, profenofos, promecarb, propoxur, prothiofos, prothoate, pymetrozine, pyraclofos, pyresmethrin, pyrethrum, pyridaben, pyridathion, pyrimidifen, pyriproxyfen,

quinalphos,

ribavirin,

salithion, sebufos, selamectin, silafluofen, spinosad, sulfotep, sulprofos,

tau-fluvalinate, tebufenozide, tebufenpyrad, tebupirimiphos, teflubenzuron, tefluthrin, temephos, temivinphos, terbufos, tetrachlorvinphos, theta-cypermethrin, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb,

vamidothion, vaniliprole, Verticillium lecanii,

YI 5302,

zeta-cypermethrin, zolaprofos,

(1R-cis)-[5-(phenylmethyl)-3-furanyl]-methyl-3-[(dihydro-2-oxo-3(2H)-furanylidene)-methyl]-2,2-dimethylcyclopropanecarboxylate,

(3-phenoxyphenyl)-methyl-2,2,3,3-tetramethylcyclopropanecarboxylate,

1-[(2-chloro-5-thiazolyl)methyl]tetrahydro-3,5-dimethyl-N-nitro-1,3,5-triazine-2(1H)-imine,

2-(2-chloro-6-fluorophenyl)-4-[4-(1,1-dimethylethyl)phenyl]-4,5-dihydro-oxazole,

2-(acetyloxy)-3-dodecyl-1,4-naphthalenedione,

2-chloro-N-[[[4-(1-phenylethoxy)-phenyl]-amino]-carbonyl]-benzamide,

2-chloro-N-[[[4-(2,2-dichloro-1,1-difluoroethoxy)-phenyl]-amino]-carbonyl-]benzamide,

3-methylphenyl propylcarbamate.

4-[4-(4-ethoxyphenyl)-4-methylpentyl]-1-fluoro-2-phenoxy-benzene,

4-chloro-2-(1,1-dimethylethyl)-5-[[2-(2,6-dimethyl-4-phenoxyphenoxy)ethyl]thio]-3(2H)-pyridazinone,

4-chloro-2-(2-chloro-2-methylpropyl)-5-[(6-iodo-3-pyridinyl)methoxy]-3(2H)-pyridazinone,

4-chloro-5-[(6-chloro-3-pyridinyl)methoxy]-2-(3,4-dichlorophenyl)-3(2H)-pyridazinone,

Bacillus thuringiensis strain EG-2348,

[2-benzoyl-1-(1,1-dimethylethyl)-hydrazinobenzoic acid,

2,2-dimethyl-3-(2,4-dichlorophenyl)-2-oxo-1-oxaspiro[4.5]dec-3-en4-yl butanoate,

[3-[(6-chloro-3-pyridinyl)methyl]-2-thiazolidinylidene]-cyanamide,

dihydro-2-(nitromethylene)-2H-1,3-thiazine-3(4H)-carboxaldehyde,

ethyl [2-[[1,6-dihydro-6-oxo-1-(phenylmethyl)-4-pyidazinyl]oxy]ethyl]-carbamate,

N-(3,4,4-trifluoro-1-oxo-3-butenyl)-glycine,

N-(4-chlorophenyl)-3-[4-(difluoromethoxy)phenyl]-4,5-dihydro-4-phenyl-1H-pyrazole-1-carboxamide,

N-[(2-chloro-5-thiazolyl)methyl]-N′-methyl-N″-nitro-guanidine,

N-methyl-N′-(1-methyl-2-propenyl)-1,2-hydrazinedicarbothioamide,

N-methyl-N′-2-propenyl-1,2-hydrazinedicarbothioamide,

O,O-diethyl [2-(dipropylamino)-2-oxoethyl]-ethylphosphoramidothioate

Mixtures with other known active compounds such as herbicides or with fertilizers and growth regulators are also possible.

When used as insecticides, the active compound combinations according to the invention can furthermore be present in their commercially available formulations and in the use forms, prepared from these formulations, as a mixture with synergists. Synergists are compounds which increase the action of the active compounds, without it being necessary for the synergist added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and stored-product pests, the active compound combinations are distinguished by an excellent residual action on wood and clay as well as good stability to alkali on limed substrates.

The active compound combinations according to the invention are not only active against plant pests, hygiene pests and stored-product pests, but also, in the veterinary medicine sector, against animal parasites (ectoparasites) such as hard ticks, soft ticks, mange mites, harvest mites, flies (stinging and licking), parasitizing fly larvae, lice, head lice, bird lice and fleas. These parasites include:

From the order of the Anoplurida, for example, Haematopinus spp., Linognathus spp., Pediculus spp., Phtirus spp., Solenopotes spp.

From the order of the Mallophagida and the suborders Amblycerina and Ischnocerina, for example, Trimenopon spp., Menopon spp., Trinoton spp., Bovicola spp., Wemeckiella spp., Lepikentron spp., Damalina spp., Trichodectes spp., Felicola spp.

From the order Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.

From the order of the Siphonapterida, for example, Pulex spp., Ctenocephalides spp., Xenopsylla spp., Ceratophyllus spp.

From the order of the Heteropterida, for example, Cimex spp., Triatoma spp., Rhodnius spp., Panstrongylus spp.

From the order of the Blattarida, for example, Blatta orientalis, Periplaneta americana, Blattella germanica, Supella spp.

From the subclass of the Acaria (Acarida) and the orders of the Meta- and Mesostigmata, for example, Argas spp., Omithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp., Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp.,. Chorioptes spp., Otodectes spp., Sarcoptes spp., Notoedres spp., Knemidocoptes spp., Cytodites spp., Laminosioptes spp.

The active compound combinations according to the invention are also suitable for controlling arthropods which attack agricultural livestock such as, for example, cattle, sheep, goats, horses, pigs, donkeys, camels, buffaloes, rabbits, chickens, turkeys, ducks, geese, honey-bees, other domestic animals such as, for example, dogs, cats, caged birds, aquarium fish and so-called experimental animals such as, for example, hamsters, guinea pigs, rats and mice. By controlling these arthropods, cases of death and reductions in productivity (for meat, milk, wool, hides, eggs, honey and the like) should be diminished, so that more economical and simpler animal husbandry is possible by the use of the active compound combinations according to the invention.

The active compound mixtures according to the invention are used in the veterinary sector in a known manner by enteral administration in the form of, for example, tablets, capsules, potions, drenches, granules, pastes, boluses, the feed-through method, suppositories, by parenteral administration such as, for example, by injections (intramuscularly, subcutaneously, intravenously, intraperitoneally and the like), implants, by nasal administration, by dermal administration in the form of, for example, immersing or dipping, spraying, pouring-on, spotting-on, washing, dusting, and with the aid of active-compound-comprising moulded articles such as collars, ear tags, tail tags, limb bands, halters, marking devices and the like.

When used for cattle, poultry, domestic animals and the like, the active compound mixtures can be applied as formulations (for example powders, emulsions, flowables) comprising the active compounds in an amount of 1 to 80% by weight, either directly or after 100- to 10 000-fold dilution, or they may be used as a chemical dip.

Moreover, it has been found that the active compound mixtures according to the invention show a potent insecticidal action against insects which destroy industrial materials.

The following insects may be mentioned by way of example and with preference, but not by way of limitation:

Beetles Such as

Hylotrupes bajulus, Chlorophorus pilosis, Anobium punctatum, Xestobium rufovillosum, Ptilinus pecticomis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec., Dinoderus minutus.

Dermapterans Such as

Sirex juvencus, Urocerus gigas, Urocerus gigas taignus, Urocerus augur.

Termites Such as

Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus.

Bristle-tails such as Lepisma saccharina.

Industrial materials in the present context are understood as meaning non-living materials such as, preferably, polymers, adhesives, glues, paper and board, leather, wood, timber products and paints.

The material which is to be protected from insect attack is very particularly preferably wood and timber products.

Wood and timber products which can be protected by the composition according to the invention, or mixtures comprising it, are to be understood as meaning, for example:

construction timber, wooden beams, railway sleepers, bridge components, jetties, vehicles made of wood, boxes, pallets, containers, telephone poles, wood lagging, windows and doors made of wood, plywood, chipboard, joinery, or timber products which quite generally are used in house construction or building joinery.

The active compounds can be used as such, in the form of concentrates or generally customary formulations such as powders, granules, solutions, suspensions, emulsions or pastes.

The abovementioned formulations can be prepared in a manner known per se, for example by mixing the active compounds with at least one solvent or diluent, emulsifier, dispersant and/or binder or fixative, water repellant, if desired desiccants and UV stabilizers, and if desired colorants and pigments and other processing auxiliaries.

The insecticidal compositions or concentrates used for protecting wood and timber products comprise the active compound according to the invention in a concentration of 0.0001 to 95% by weight, in particular 0.001 to 60% by weight.

The amount of composition or concentrate employed depends on the species and the abundance of the insects and on the medium. The optimal quantity to be employed can be determined in each case by test series upon application. In general, however, it will suffice to employ 0.0001 to 20% by weight, preferably 0.001 to 10% by weight, of the active compound, based on the material to be protected.

A suitable solvent and/or diluent is an organochemical-solvent or solvent mixture and/or an oily or oil-type organochemical solvent or solvent mixture of low volatility and/or a polar organochemical solvent or solvent mixture and/or water and, if appropriate, an emulsifier and/or wetter.

Organochemical solvents which are preferably employed are oily or oil-type solvents with an evaporation number of above 35 and a flash point of above 30° C., preferably above 45° C. Such oily and oil-type solvents which are insoluble in water and of low volatility and which are used are suitable mineral oils or their aromatic fractions or mineral-oil-containing solvent mixtures, preferably white spirit, petroleum and/or alkylbenzene.

Mineral oils with a boiling range of 170 to 220° C., white spirit with a boiling range of 170 to 220° C., spindle oil with a boiling range of 250 to 350° C., petroleum and aromatics with a boiling range of 160 to 280° C., oil of turpentine, and the like are advantageously used.

In a preferred embodiment, liquid aliphatic hydrocarbons with a boiling range of 180 to 210° C. or high-boiling mixtures of aromatic and aliphatic hydrocarbons with a boiling range of 180 to 220° C. and/or spindle oil and/or monochloronaphthalene, preferably α-monochloronaphthalene, are used.

The organic oily or oil-type solvents of low volatility and with an evaporation number of above 35 and a flash point of above 30° C., preferably above 45° C., can be replaced in part by organochemical solvents of high or medium volatility, with the proviso that the solvent mixture also has an evaporation number of above 35 and a flash point of above 30° C., preferably above 45° C., and that the mixture is soluble or emulsifiable in this solvent mixture.

In a preferred embodiment, some of the organochemical solvent or solvent mixture is replaced by an aliphatic polar organochemical solvent or solvent mixture. Aliphatic organochemical solvents which contain hydroxyl and/or ester and/or ether groups are preferably used, such as, for example, glycol ethers, esters or the like.

Organochemical binders used for the purposes of the present invention are the synthetic resins and/or binding drying oils which are known per se and which can be diluted in water and/or dissolved or dispersed or emulsified in the organochemical solvents employed, in particular binders composed of, or comprising, an acrylate resin, a vinyl resin, for example polyvinyl acetate, polyester resin, polycondensation or polyaddition resin, polyurethane resin, alkyd resin or modified alkyd resin, phenol resin, hydrocarbon resin such as indene/coumarone resin, silicone resin, drying vegetable and/or drying oils and/or physically drying binders based on a natural and/or synthetic resin.

The synthetic resin employed as binder can be employed in the form of an emulsion, dispersion or solution. Bitumen or bituminous substances may also be used as binders, in amounts of up to 10% by weight. In addition, colorants, pigments, water repellants, odour-masking agents, and inhibitors or anticorrosive agents and the like, all of which are known per se, can be employed.

In accordance with the invention, the composition or the concentrate preferably comprises, as organochemical binders, at least one alkyd resin or modified alkyd resin and/or a drying vegetable oil. Alkyd resins which are preferably used in accordance with the invention are those with an oil content of over 45% by weight, preferably 50 to 68% by weight.

Some or all of the abovementioned binder can be replaced by a fixative (mixture) or plasticizer (mixture). These additives are intended to prevent volatilization of the active compounds, and also crystallization or precipitation. They preferably replace 0.01 to 30% of the binder (based on 100% of binder employed).

The plasticizers are from the chemical classes of the phthalic esters, such as dibutyl phthalate, dioctyl phthalate or benzyl butyl phthalate, phosphoric esters such as tributyl phosphate, adipic esters such as di-(2-ethylhexyl)-adipate, stearates such as butyl stearate or amyl stearate, oleates such as butyl oleate, glycerol ethers or higher-molecular-weight glycol ethers, glycerol esters and p-toluenesulphonic esters.

Fixatives are based chemically on polyvinyl alkyl ethers such as, for example, polyvinyl methyl ether, or ketones such as benzophenone and ethylenebenzophenone.

Other suitable solvents or diluents are, in particular, water, if appropriate as a mixture with one or more of the abovementioned organochemical solvents or diluents, emulsifiers and dispersants.

Particularly effective timber protection is achieved by industrial-scale impregnating processes, for example the vacuum, double-vacuum or pressure processes.

The ready-to-use compositions can also comprise other insecticides, if appropriate, and also one or more fungicides, if appropriate.

Possible additional mixing partners are, preferably, the insecticides and fungicides mentioned in WO 94/29 268. The compounds mentioned in this document are an explicit constituent of the present application.

Especially preferred mixing partners which may be mentioned are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyfenozide and triflumuron, and also fungicides, such as epoxyconazole, hexaconazole, azaconazole, propiconazole, tebuconazole, cyproconazole, metconazole, imazalil, dichlorfluanid, tolylfluanid, 3-iodo-2-propinyl -butyl carbamate, N-octyl-isothiazolin-3-one and 4,5-dichloro-Noctylisothiazolin-3-one.

The mixtures according to the invention can at the same time be employed for protecting objects which come into contact with saltwater or brackish water, such as hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent stops in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the mixtures according to the invention, on their own or in combination with other active compounds, have an outstanding antifouling action.

Using the mixtures according to the invention on their own or in combination with other active compounds allows the use of heavy metals such as, for example, in bis(trialkyltin) sulphides, tri-n-butyltin lautate, tri-n-butyltin chloride, copper(I) oxide, triethyltin chloride, tri-n-butyl(2-phenyl4-chlorophenoxy)tin, tributyltin oxide, molybdenum disulphide, antimony oxide, polymeric butyl titanate, phenyl-(bispyridine)-bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylziric ethylenebisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferable suitable components in combinations with the antifouling compositions according to the invention are:

algicides such as

2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;

fungicides such as

benzo[b]thiophenecarboxylic acid cyclohexylamide S,S-dioxide, dichlofluanid, fluorfolpet, 3-iodo-2-propinyl butylcarbamate, tolylfluanid and azoles such as azaconazole, cyproconazole, epoxyconazole, hexaconazole, metconazole, propiconazole and tebuconazole;

molluscicides such as

fentin acetate, metaldehyde, methiocarb, niclosamid, thiodicarb and trimethacarb;

or conventional antifouling active compounds such as

4,5-dichloro-2-octyl4-isothiazolin-3-one, diiodomethylparatryl sulphone, 2-(N,Ndimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulphonyl)-pyridine, 2,4,5,6-tetrachloroisophthalonitrile, tetramethylthiuram disulphide and 2,4,6-trichlorophenylmaleiimide.

The antifouling compositions used comprise the active compound according to the invention of the combinations according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as colophonium to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compound mixtures are also suitable for controlling animal pests, in particular insects, arachnids and mites, which are found in enclosed spaces such as, for example, dwellings, factory halls, offices, vehicle cabins and the like. They can be employed on their own or in combination with other active compounds an excipients in domestic insecticide products for controlling these pests. They are active against sensitive and resistant species and against all developmental stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Omithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodnius prolixus, Triatoma infestans.

In the field of household insecticides, they are used alone or in combination with other suitable active compounds, suh as phosphonic acid esters, carbamates, pyrethroids, growth regulators or active compounds from other known classes of insecticides.

They are used as aerosols, pressureless spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The terms “parts”, “parts of plants” and “plant parts” have been explained above.

Particularly preferably, plants of the plant cultivars which are in each case commercially available or in use are treated according to the invention. Plant cultivars are to be understood as meaning plants having certain properties (“traits”) which have been obtained by conentional breeding, by mutagenesis or by recombinant DNA techniques. This can be varieties, bio- and genotypes

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferred and to be treated according to the invention include all plants which, in the genetic modification, received genetic material which imparts particularly advantageous useful traits to these plants. Examples of such traits are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are particularly emphasized are the increased defence of the plants against insects by toxins formed in the plants, in particular those formed by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits that are also particularly emphasized are the increased defence of the plants to fungi, bacteria and viruses by systemic acquired resistance (SAR), systemin, phytoalexins, elicitors, and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes in question which impart the desired traits can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and Newleaf® (potato). Examples of herbicide-tolerant plants which may be mentioned are maize varieties, cotton varieties and soya bean varieties which are sold under the trade names Roundup Ready®) (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these or still-to-be-developed genetic traits, which plants will be developed and/or marketed in the future.

The plants listed can be treated according to the invention in a particularly advantageous manner with the active compound mixtures according to the invention.

The preferred ranges stated above for the mixtures also apply to the treatment of these plants. Particular emphasis is given to the treatment of plants with the compounds or mixtures specifically mentioned in the present text.

### USE EXAMPLES

Formula for Calculating the Kill Rate for a Combination of Two Active Compounds

The expected action for a given combination of two active compounds can be calculated as follows (cf. Colby, S. R., “Calculating Synergistic and Antagonistic Responses of Herbicide Combinations”, Weeds 15, pages 20-22, 1967):

if

X is the kill rate, expressed as a percentage of the untreated control, when employing active compound A at an application rate of m ppm,

Y is the kill rate, expressed as a percentage of the untreated control, when employing active compound B at an application rate of n ppm and

E is the kill rate, expressed as a percentage of the untreated control, when employing active compounds A and B at application rates of m and n ppm,

then

$$E = X + Y - \frac{X \cdot Y}{100}$$

If the actual insecticidal kill rate exceeds the calculated value, the action of the combination is superadditive, i.e. a synergistic effect is present. In this case, the actually observed kill rate must exceed the value calculated using the above formula for the expected kill rate (E).

### Example A

Phaedon Larvae Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with larvae of the mustard beetle (Phaedon cochleariae) while the leaves are still moist.

After the desired period of time, the kill in % is determined. 100% means that all beetle larvae have been killed; 0% means that none of the beetle larvae have been killed. The kill rates determined are calculated using Colby's formula (see sheet 1).

In this test, the following active compound combination according to the present application showed a synergistically enhanced activity compared to the active compounds applied individually:

TABLE A
sheet 1
Plant-damaging insects
Phaedon larvae test

| | Concentration of active | Kill rate | |
|---|---|---|---|
| Active compounds | compounds in ppm | in % after 3 days | |
| indoxacarb | 0.5 | 20 | |
| imidacloprid | 3 | 0 | |
| | | found* | calc.** |
| indoxacarb + imidacloprid | 0.5 + 3 | 100 | 20 |
| according to the | | | |
| invention | | | |

found* = activity found
calc.** = activity calculated using Colby's formula

TABLE A
sheet 2
Plant-damaging insects
Phaedon larvae test

| | Concentration of active | Kill rate | |
|---|---|---|---|
| Active compounds | compounds in ppm | in % after 6 days | |
| indoxacarb | 0.5 | 45 | |
| thiacloprid | 3 | 0 | |
| | | found* | calc.** |
| indoxacarb + thiacloprid | 0.5 + 3 | 85 | 45 |
| according to the | | | |
| invention | | | |

found* = activity found
calc.** = activity calculated using Colby's formula

### Example B

Plutella Test (Normally Sensitive)

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with normally sensitive caterpillars of the diamondback moth (Plutella xylostella) while the leaves are still moist.

After the desired period of time, the kill in % is determined. 100% means that all caterpillars have been killed; 0% means that none of the caterpillars have been killed. The kill rates determined are calculated using Colby's formula (see sheet 1).

In this test, the following active compound combination according to the present application showed a synergistically enhanced activity compared to the active compounds applied individually:

TABLE B
Plant-damaging insects
Plutella test (normally sensitive)

| | Concentration of active | Kill rate | |
|---|---|---|---|
| Active compounds | compounds in ppm | in % after 6 days | |
| indoxacarb | 0.1 | 65 | |
| thiacloprid | 3 | 0 | |
| | | found* | calc.** |
| indoxacarb + thiacloprid | 0.1 + 3 | 100 | 65 |
| according to the | | | |
| invention | | | |

found* = activity found
calc.** = activity calculated using Colby's formula

### Example C

Plutella Test (Resistant)

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with resistant caterpillars of the diamondback moth (Plutella xylostella) while the leaves are still moist.

After the desired period of time, the kill in % is determined. 100% means that all caterpillars have been killed; 0% means that none of the caterpillars have been killed. The kill rates determined are calculated using Colby's formula (see sheet 1).

In this test, the following active compound combination according to the present application showed a synergistically enhanced activity compared to the active compounds applied individually:

TABLE C
Plant-damaging insects
Plutella test (resistant)

| | Concentration of active | Kill rate | |
|---|---|---|---|
| Active compounds | compounds in ppm | in % after 6 days | |
| indoxacarb | 0.1 | 55 | |
| thiacloprid | 3 | 0 | |
| | | found* | calc.** |
| indoxacarb + thiacloprid | 0.1 + 3 | 85 | 55 |
| according to the | | | |
| invention | | | |

found* = activity found
calc.** = activity calculated using Colby's formula

### Example D

Spodoptera frugiperda Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the armyworm (Spodoptera frugiperda) while the leaves are still moist.

After the desired period of time, the kill in % is determined. 100% means that all caterpillars have been killed; 0% means that none of the caterpillars have been killed. The kill rates determined are calculated using Colby's formula (see sheet 1).

In this test, the following active compound combination according to the present application showed a synergistically enhanced activity compared to the active compounds applied individually:

TABLE D
Plant-damaging insects
Spodoptera frugiperda test

| | Concentration of active | Kill rate | |
|---|---|---|---|
| Active compounds | compounds in ppm | in % after 6 days | |
| indoxacarb | 0.02 | 0 | |
| imidacloprid | 0.6 | 0 | |
| | | found* | calc.** |
| indoxacarb + imidacloprid | 0.02 + 0.6 | 100 | 0 |
| according to the | | | |
| invention | | | |

found* = activity found
calc.** = activity calculated using Colby's formula

### Example E

Heliothis virescens Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Soya bean shoots (Glycine max) are treated by being dipped into the preparation of active compound of the desired concentration and are populated with Heliothis virescens caterpillars while the leaves are still moist.

After the desired period of time, the kill in % is determined. 100% means that all caterpillars have been killed; 0% means that none of the caterpillars have been killed. The kill rates determined are calculated using Colby's formula (see sheet 1).

In this test, the following active compound combination according to the present application showed a synergistically enhanced activity compared to the active compounds applied individually:

TABLE E
Plant-damaging insects
Heliothis virescens test

| | Concentration | Kill rate | |
|---|---|---|---|
| | of active | in % after | |
| Active compounds | compounds in ppm | 3 days | |
| indoxacarb | 0.1 | 0 | |
| imidacloprid | 3 | 15 | |
| | | found* | calc.** |
| indoxacarb + imidacloprid | 0.1 + 3 | 100 | 15 |
| according to the | | | |
| invention | | | |

found* = activity found
calc.** = activity calculated using Colby's formula

Formula for Calculating the Synergistic Effect of a Combination of Two Active Compounds

The expected action for a given combination of two active compounds can be calculated as follows (cf. Carpenter, C. S., “Mammalian Toxicity of 1-Naphthyl-N-methylcarbamate [Sevin Insecticide]”, Agricultural and Food Chemistry, Vol. 9, No. 1, pages 30-39, 1961):

If

Pa is the percentage of active compound A in the mixture,

Pb is the percentage of active compound B in the mixture,

LC_{50 (or 95)}a is the concentration at which 50% (or 95%) of the animals treated with active compound A are killed and

LC_{50 (or 95)}b is the concentration at which 50% (or 95%) of the animals treated with active compound B are killed,

ps then the expected

$${LC}_{50 ( or 95 )} ( comb . ) = \frac{1}{\frac{Pa}{{LC}_{50 ( or 95 )} a} + \frac{Pb}{{LC}_{50 ( or 95 )} b}}$$

If the calculated LC50(or 95) is higher than the actual value and above the confidence interval, the action of the combination is superadditive, i.e. a synergistic effect is present.

### Example A

Phaedon Larvae Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with larvae of the mustard beetle (Phaedon cochleariae) while the leaves are still moist.

After the desired period of time, the kill in % is determined. 100% means that all beetle larvae have been killed; 0% means that none of the beetle larvae have been killed. The kill rates determined are calculated using Carpenter's formula (see sheet

In this test, the following active compound combination according to the present application showed a synergistically enhanced activity compared to the active compounds applied individually:

TABLE A
Plant-damaging insects
Phaedon larvae test

| Active compounds | | LC₅₀ after 6 days | |
|---|---|---|---|
| indoxacarb | | 0.1 | ppm |
| known | | | |
| clothianidin (X) | | 2.372 | ppm |
| known | | | |
| indoxacarb + clothianidin (X) (1:6) | calc.** | 0.558 | ppm |
| according to the invention | found* | 0.1 | ppm |

found* = activity found
calc.** = activity calculated using Carpenter's formula

### Example B

Plutella Test, Sensitive Strain

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the diamondback moth (Plutella xylostella, sensitive strain) while the leaves are still moist.

After the desired period of time, the kill in % is determined. 100% means that all caterpillars have been killed; 0% means that none of the caterpillars have been killed. The kill rates determined are calculated using Carpenter's formula (see sheet 1).

In this test, the following active compound combination according to the present application showed a synergistically enhanced activity compared to the active compounds applied individually:

TABLE B
Plant-damaging insects
Plutella test, sensitive strain

| Active compounds | | LC₉₅ after 6 days | |
|---|---|---|---|
| indoxacarb | | 0.531 | ppm |
| known | | | |
| clothianidin (X) | | 26.037 | ppm |
| known | | | |
| indoxacarb + clothianidin (X) (1:6) | calc.** | 3.311 | ppm |
| according to the invention | found* | 0.322 | ppm |

found* = activity found
calc.** = activity calculated using Carpenter's formula

### Example C

Plutella Test, Resistant Strain

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration. Cabbage leaves (Brassica oleracea) are treated by being dipped into the preparation of active compound of the desired concentration and are populated with caterpillars of the diamondback moth (Plutella xylostella, resistant strain) while the leaves are still moist.

After the desired period of time, the kill in % is determined. 100% means that all caterpillars have been killed; 0% means that none of the caterpillars have been killed. The kill rates determined are calculated using Carpenter's formula (see sheet 1).

In this test, the following active compound combination according to the present application showed a synergistically enhanced activity compared to the active compounds applied individually:

TABLE C
Plant-damaging insects
Plutella test, resistant strain

| Active compounds | | LC₉₅ after 6 days | |
|---|---|---|---|
| indoxacarb | | 0.234 | ppm |
| known | | | |
| clothianidin (X) | | 50.722 | ppm |
| known | | | |
| indoxacarb + clothianidin (X) (1:6) | calc.** | 1.592 | ppm |
| according to the invention | found* | 0.214 | ppm |

found* = activity found
calc.** = activity calculated using Carpenter's formula

### Example D

Spodoptera frugiperda Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the armyworm (Spodoptera frugiperda) while the leaves are still moist.

After the desired period of time, the kill in % is determined. 100% means that all caterpillars have been killed; 0% means that none of the caterpillars have been killed. The kill rates determined are calculated using Carpenter's formula (see sheet 1).

In this test, the following active compound combination according to the present application showed a synergistically enhanced activity compared to the active compounds applied individually:

TABLE D
Plant-damaging insects
Spodoptera frugiperda test

| Active compounds | | LC₅₀ after 6 days | |
|---|---|---|---|
| indoxacarb | | 0.136 | ppm |
| known | | | |
| clothianidin | | 0.662 | ppm |
| known | | | |
| indoxacarb + clothianidin (X) (1:30) | calc.** | 0.589 | ppm |
| according to the invention | found* | 0.027 | ppm |

found* = activity found
calc.** = activity calculated using Carpenter's formula

1. Mixtures, comprising one or more sodium ion channel effectors and the compound (Z)-3-(6-chloro-3-pyridylmethyl)-1,3-thiazolidin-2-ylidene-cyanamide or the compound (E)-1-(2-chloro-1,3-thiazol-5-ylmethyl)-3-methyl-2-nitroguanidine or the compound 1-[(6-chloro-3-pyridinyl)-methyl]-N-nitro-2-imidazolidinimine.

2. Mixture, comprising the active compounds indoxacarb of the formula 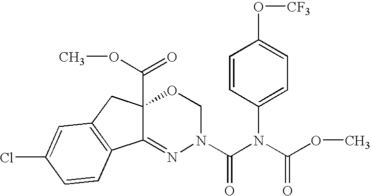
and clothianidin of the formula (X)

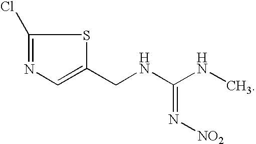

3. Mixture, comprising the active compounds indoxacab of the formula 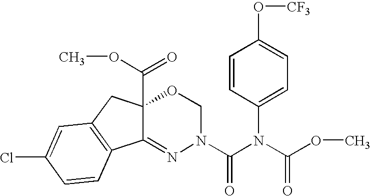
and thiacloprid of the formula (IX)

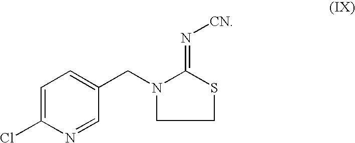

4. Mixture, comprising the active compounds indoxacarb of the formula 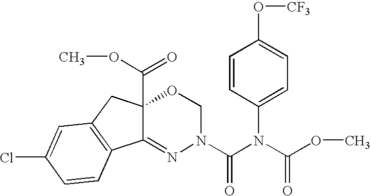
and imidacloprid of the formula (XI)

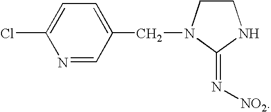

5. Mixtures according to any of claims 1 to 4 in which the ratio of the Na ion channel effector to the compound (Z)-3-(6-chloro-3-pyridylmethyl)-1,3-thiazolidin-2-ylidenecyanamide or the compound (E)-1-(2-chloro-1,3-thiazol-5-ylmethyl)-3-methyl-2-nitroguanidine or the compound 1-[(6-chloro-3-pyridinyl)methyl]-N-nitro-2-imidazolidinimine is from 1:0.1 to 1:100.

6. Use of mixtures according to any of claims 1 to 5 for controlling animal pests.
